(19) United States
(12) Patent Application Publication
(10) Pub. No.: US 2024/0317664 A1
(43) Pub. Date: Sep. 26, 2024

(54) PHENANTHRENE-BASED COMPOUND HAVING HIGH REFRACTIVE INDEX AND PREPARATION METHOD THEREFOR

(71) Applicant: Research & Business Foundation Sungkyunkwan University, Suwon-si (KR)

(72) Inventors: Younchul KIM, Seongnam-si (KR); Jinho KIM, Incheon (KR); Subeen KIM, lncheon (KR)

(73) Assignee: Research & Business Foundation Sungkyunkwan University, Suwon-si (KR)

(21) Appl. No.: 18/613,732

(22) Filed: Mar. 22, 2024

(30) Foreign Application Priority Data
Mar. 23, 2023 (KR) 10-2023-0038271

(51) Int. Cl. C07C 45/67; C07C 29/38; C07C 37/055; C07C 39/17; C07C 49/613

(52) U.S. Cl. CPC C07C 45/673; C07C 29/38; C07C 37/055; C07C 39/17; C07C 49/613; C07C 2603/26

(57) ABSTRACT

Disclosed herein are a phenanthrene-based compound with a high-refractive index and a preparation method therefor. The compound has a phenanthrene-based complex cardo structure and can be used as a monomer in optical resins requiring a refractive index of 1.7 or higher. Due to the phenanthrene-based complex cardo structure of the compound, the fluidity of the molecular chains can be maximally suppressed, allowing for the production of resins with excellent thermal stability, characterized by a high glass transition temperature.

### BACKGROUND OF THE INVENTION

### 1. Field of the Invention

This invention was carried out with the support of the Ministry of Trade, Industry and Energy of the Republic of Korea, under the unique project number 1415179963, project number 20013223. The project management organization for this task is the Korea Industrial Technology Evaluation Management Institute, with the research project title “Material Component Technology Development” and the research topic name “Development of Thermoplastic Optical Resin with Refractive Index of 1.65 or Higher for Smart Device Optical Lenses and Light-Shielding Agent with Optical Density of 6.5 or Higher.” The executing institution is Kukbo Chemical Co., Ltd., and the research period was from Jan. 1, 2022, to Dec. 31, 2022.

Additionally, this invention was also supported by the Ministry of Trade, Industry and Energy of the Republic of Korea, under the unique project number 1415178757, project number 20013794. The project management organization for this task is the Korea Industrial Technology Evaluation Management Institute, with the research project title “Industrial Technology Hub Center Nurturing Pilot Project” and the research topic name “Composite Material Simultaneous Design Industrial Technology Hub Center.” The executing institution is the Industry-University Cooperation Foundation of Sungkyunkwan University, and the research period was from Jan. 1, 2022, to Dec. 31, 2022.

This patent application claims priority to and the benefit of Korean Patent Application No. 10-2023-0038271 filed with the Korean Intellectual Property Office on Mar. 23, 2023, the disclosure of which is incorporated herein by reference.

The present disclosure relates to a phenanthrene-based compound with a high refractive index and a preparation method therefor. More specifically, the present disclosure relates to a compound with a high refractive index that has a phenanthrene-based complex cardo structure and can be used in optical resins requiring a refractive index of 1.7 or higher.

### 2. Description of the Prior Art

Conventionally, monomers with improved refractive indices, such as compounds represented by the following Chemical Formulas A to C, have been used to manufacture optical resins with high refractive indices. However, it is difficult to apply these compounds to uses requiring a refractive index of 1.7 or higher.

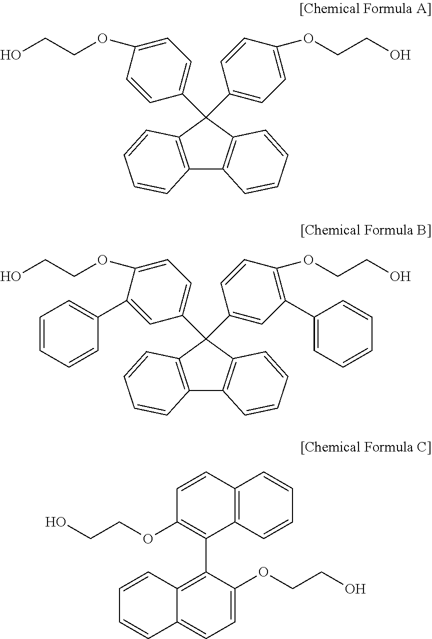 [SMILES: OCCOc1ccc(C2(c3ccc(OCCO)cc3)c3ccccc3-c3ccccc32)cc1 ; OCCOc1ccc(C2(c3ccc(OCCO)c(-c4ccccc4)c3)c3ccccc3-c3ccccc32)cc1-c1ccccc1 ; OCCOc1ccc2ccccc2c1-c1c(OCCO)ccc2ccccc12]

Accordingly, there has been a need to develop novel compounds that possess functional groups applicable to resins such as polyester, polycarbonate, polyurethane, polyolefin, epoxy, acryl, and the like and have higher refractive indices than the commercially available monomers for optical resins.

### SUMMARY OF THE INVENTION

Leading to the present disclosure, intensive and thorough research conducted by the present inventors culminated in preparing phenanthrene-based compounds with high refractive indices applicable to optical resins.

Therefore, the present disclosure aims to provide phenanthrene-based compounds with high refractive indices and preparation method therefor.

Optical resins like those in the present disclosure are used in various industrial fields such as displays, semiconductors, solar energy, cameras, optical fibers, sensors, etc. In particular, optical resins are expanding as a replacement for conventional glass materials in recent IT devices due to their advantages of being lightweight, cost competitiveness, and high impact resistance. However, the development of high-refractive-index monomers and resins that can overcome the low refractive index and thermal stability issues of current optical resins continues to be demanded. Especially, high-refractive materials can broaden the viewing angles of displays, reduce the thickness of optical lenses, and enhance light extraction efficiency, playing a crucial role in the clarity of images and energy efficiency. Hence, high-refractive materials exhibit rapid progress in their industrial application expansion.

Leading to the present disclosure, intensive and thorough research efforts, devoted by the present inventors, towards developing novel compounds with high refractive indices applicable to optical resins resulted in the finding that phenanthrene-based compounds exhibit high refractive indices and are suitable for use in optical resins.

An aspect of the present disclosure provides a phenanthrene-based compound having the chemical structure represented by the following Chemical Formula 1:

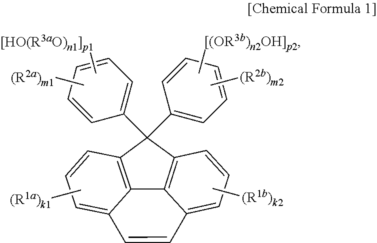

wherein R^{1a}, R^{1b}, R^{2a}, and R^{2b} are each a substituent bonded
to the phenanthrene or phenyl moieties,
R^{3a} and R^{3b} are each an alkylene;
k1 and k2 are each independently an integer of 0 to 4;
m1 and m2 are each independently an integer of 0 to 3;
n1 and n2 are each independently an integer of 0 or 1 or higher;
p1 and p2 are each independently an integer of 1 or higher,
with a proviso that m1+p1 and m2+p2 are each independently an integer of 1 to 4.

### DETAILED DESCRIPTION OF THE EXEMPLARY EMBODIMENTS

The compound of the present disclosure has a phenanthrene-based complex cardo structure and is a high-refractive-index monomer that can be used in optical resins requiring a refractive index of 1.7 or higher.

As used herein, the term “cardo” compound refers to a compound having a cyclic side group pendent to the backbone thereof. With the structural feature of bulky lateral groups pendent to the polymer backbone thereof, cardo compounds undergo significant rotational hindrance. Furthermore, the addition of the phenanthrene structure imparts a greater high refractive index as well as very high heat resistance (high glass transition temperature) and excellent processability to the cardo compounds.

In an embodiment of the present disclosure, the substituents represented by R^{1a} and R^{1b} are, for the most part, alkyls, but with no limitations thereto. The alkyl may be a C1 to C6 alkyl (e.g., C1-4 alkyl, especially methyl), as exemplified by methyl, ethyl, propyl, isopropyl, butyl, t-butyl, etc. R^{1a} and R^{1b} may be different or same. Furthermore, the bonding position (substitution position) of R^{1a} or R^{1b} on the benzene rings constituting the phenanthrene skeleton is not particularly limited. The substitution numbers k1 and k2 are 0 or 1. Moreover, the substitution numbers k1 and k2 may be different or same and are generally identical.

Examples of the substituents R^{2a} and R^{2b} include hydrocarbons such as alkyls (C1 to C20 alkyl, e.g. methyl, ethyl, propyl, isopropyl, butyl, s-butyl, t-butyl, etc., preferably alkyl of C1 to C8, more preferably alkyl of C1 to C6), cycloalkyls (C5 to C10 cycloalkyl, e.g., cyclopentyl, cyclohexyl, etc., preferably C5-8 cycloalkyl, more preferably C5-6 cycloalkyl), aryls (C6 to C10 aryl, e.g., phenyl and alkylphenyl (methylphenyl, methylphenyl, etc.), preferably C6 to C8 aryl, especially phenyl), and aralkyls (C6-10 aryl-C1-C4 alkyl containing, benzyl, phenethyl, etc.); alkoxy (C1-C4 alkoxy such as methoxy, etc.); acyl (C1-C6 acyl such as acetyl); alkoxycarbonyl (C1-C4 alkoxycarbonyl such as methoxycarbonyl, etc.); halogen atoms (fluorine atom, chlorine atom, etc.); nitro; and cyano. The substituent R^{2a} or R^{2b} may be preferably an alkyl (C1 to C6 alkyl), a cycloalkyl (C5 to C8 cycloalkyl), an aryl (C6 to C10 aryl), or an aralkyl (C6 to C8 aryl-C1 to C2 alkyl), with particular preference for C1 to C6 alkyl (e.g., C1 to C4 alkyl), C1 to C4 alkoxy, and C6 to C8 aryl. The substituents R^{2a} and R^{2b} may each or together be a benzene ring. In addition, R^{2a} and R^{2b} may be same or different and generally be identical. In addition, R^{2a} or R^{2b} may be same or different on the same benzene ring moiety. No particular limitations are imparted to the bonding positions of the substituent R^{2a} or R^{2b}.

m1 and m2, which account for numbers of the hydroxyl radical-containing substituents, may each be 0-2 and preferably 0 or 1. In addition, the substitution number m1 and m2 may be same or different and are, for the most part, identical.

Examples of the alkylene accounting for R^{3a} and R^{3b} include, but are not limited to, C2 to C6 alkylene (ethylene, trimethylene, propylene, butan-1,4-diyl (tetramethylene), butan-1,2-diyl, etc.), with preference for C2 to C4 alkylene and more preference for C2 to C3 alkylene (particularly, ethylene and propylene). R^{3a} and R^{3b} may be same or different and are generally the same alkylene.

n1 and n2, which each account for a number of the substituent alkoxy, may be 0 or may range from 1 to 15 (e.g., 0 or 1-12). For example, n1 and n2 may each be 0 or 1-10 (e.g., 0 or 1-6), preferably 0 or 1-4, more preferably 0 or 1-3 (e.g., 0-2), and particularly 0 or 1. In addition, the sum of n1 and n2 (n1+n2) may be 0 or 1-30 (e.g., 0 or 1-24), for example, 0 or 1-20 (e.g., 0 or 1-12), preferably 0 or 1-8, more preferably 0 or 1-6 (e.g., 0-4), and particularly 0 or 2. In addition, when n1 or n2 is 2 or higher, the polyalkoxy (polyalkyleneoxy) radicals may be the same alkoxy or may be composed of different alkoxy radicals (for example, ethoxy and propyleneoxy), with preference for the same alkoxy.

p1 and p2, which account for numbers of the hydroxyl-containing substituents, may each be preferably 1-2 and particularly preferably 1. The sum of p1 and p2 (p1+p2) may be, for example, 2-6 and preferably 2-4 (especially 2). Moreover, p1 and p2, which may be different, are, for the most part, same. The substitution positions of the hydroxyl group-containing substituents are not particularly limited.

In an embodiment of the present disclosure, R^{3a} and R^{3b} are each independently a C2 to C4 alkylene; n1 and n2 are each independently 0 or 1, but with no limitations thereto.

In an embodiment of the present disclosure, p1 and p2 are each 1, but are not limited thereto.

In an embodiment of the present disclosure, R^{1a} and R^{1b} are each independently a C1 to C4 alkyl; k1 and k2 are each independently 0 or 1; R^{2a} and R^{2b} are each independently a C1 to C4 alkyl, a C1 to C4 alkoxy, or a C6 to C8 aryl; m1 and m2 are each independently an integer of 0 to 2, but with no limitations thereto.

In a particular embodiment of the present disclosure, the phenanthrene compound represented by Chemical Formula 1 is a compound with the chemical structure of the following Chemical Formula 2-1 or 2-2:

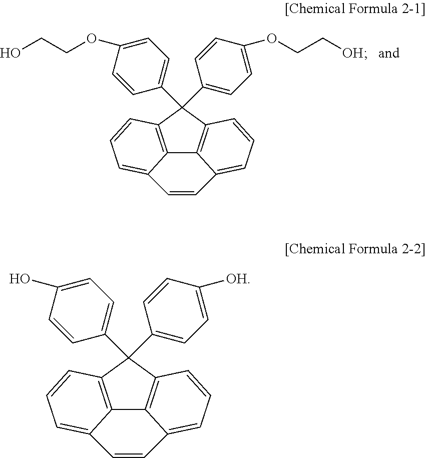 [SMILES: OCCOc1ccc(C2(c3ccc(OCCO)cc3)c3cccc4ccc5cccc2c5c34)cc1 ; "and" ; Oc1ccc(C2(c3ccc(O)cc3)c3cccc4ccc5cccc2c5c34)cc1]

According to another aspect thereof, the present disclosure provides a method for preparing a phenanthrene-based compound, the method including the steps of:
(a) oxidizing pyrene to synthesize pyrene-4,5-dione;
(b) synthesizing cyclopentaphenanthrene-4-one from pyrene-4,5-dione; and
(c) synthesizing 2,2′-(((4H-cyclopentaphenanthrene-4,4-diyl) bis (4,1-phenylene)) bis (oxy)) diethanol from cyclopentaphenanthrene-4-one.

In an embodiment of the present disclosure, the method for preparing a phenanthrene-based compound further includes the step of (d) synthesizing 4,4′-(4H-cyclopentaphenanthrene-4,4-diyl) diphenol from 2,2′-(((4H-cyclopentaphenanthrene-4,4-diyl) bis (4,1-phenylene)) bis (oxy)) diethanol.

### Step (a): Synthesis of Pyrene-4,5-Dione

In an embodiment of the present disclosure, the step (a) is set to oxidize pyrene with sodium periodate to synthesize pyrene-4,5-dione as illustrated in Reaction Scheme 1-1, below:

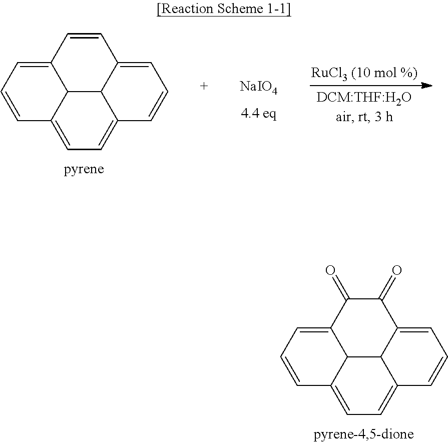

In a particular embodiment of the present disclosure, a mixture of pyrene and ruthenium chloride (RuCl₃) was dissolved in tetrahydrofuran (THF) as a solvent by stirring under a nitrogen atmosphere, and the solution was added with an aqueous NaIO₄ solution and then with dichloromethane (DCM) solvent, followed by stirring at room temperature. The stirring may be continued for 3 to 5 hours.

In a particular embodiment of the present disclosure, NaIO₄ may be added in an amount of 60 to 120 mmol, 60 to 100 mmol, 60 to 90 mmol, 60 to 88 mmol, 70 to 120 mmol, 70 to 100 mmol, 70 to 90 mmol, 70 to 88 mmol, 80 to 120 mmol, 80 to 100 mmol, 80 to 90 mmol, or 80 to 88 mmol, based on 20 mmol of pyrene, but with no limitations thereto.

In a particular embodiment of the present disclosure, the stirred solution was filtered to remove solids therefrom, followed by extraction with dichloromethane (DCM) and H₂O. The organic layer thus formed was added with MgSO₄ and filtered.

In a particular embodiment of the present disclosure, the filtrate was recrystallized at a reduced pressure to obtain pyrene-4,5-dione. The recrystallization can be achieved by adding dichloromethane (DCM) and hexane solution.

### Step (b): Synthesis of Cyclopentaphenanthrene-4-One

In an embodiment of the present disclosure, step (b) is set to react pyrene-4,5-dione with sodium hydroxide to synthesize cyclopentaphenanthrene-4-one, as illustrated by Reaction Scheme 1-2, below:

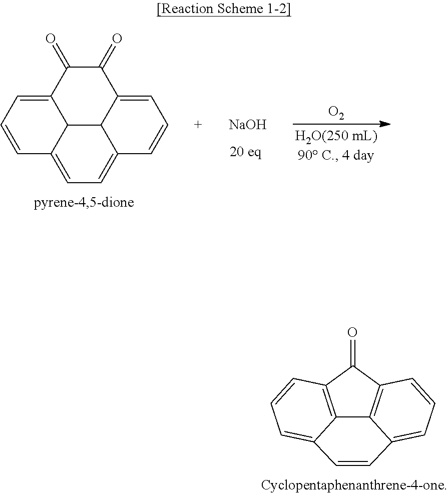 [SMILES: O=C1C(=O)C2=CC=CC3=CC=C4C=CC=C1C4C32 ; "NaOH" ; "O2" ; O=c1c2cccc3ccc4cccc1c4c32]

In a particular embodiment of the present disclosure, pyrene-4,5-dione synthesized in step (a) was reacted with NaOH in an oxygen atmosphere to synthesize cyclopentaphenanthrene-4-one.

In a particular embodiment of the present disclosure, NaOH may be added in an amount of 5 to 40 mmol, 5 to 35 mmol, 5 to 30 mmol, 5 to 25 mmol, 5 to 20 mmol, 10 to 40 mmol, 10 to 35 mmol, 10 to 30 mmol, 10 to 25 mmol, 10 to 20 mmol, 15 to 40 mmol, 15 to 35 mmol, 15 to 30 mmol, 15 to 25 mmol, 15 to 20 mmol, 20 to 40 mmol, 20 to 35 mmol, 20 to 30 mmol, or 20 to 25 mmol, based on 10 mmol of pyrene-4,5-dione, but with no limitations thereto.

The reaction may be carried out at a temperature of 70° C. to 120° C., 80° C. to 120° C., 90° C. to 120° C., 70° C. to 110° C., 80° C. to 110° C., 90° C. to 110° C., 70° C. to 100° C., 80° C. to 100° C., 90° C. to 100° C., 70° C., 80° C., or 90° C., but with no limitations thereto.

The reaction may be carried out for 2 to 6 days, 3 to 6 days, 4 to 6 days, 2 to 5 days, 3 to 5 days, 4 to 5 days, 2 to 4 days, 3 to 4 days, 3 days, 4 days, 5 days, or 6 days, but with no limitations thereto.

In a particular embodiment of the present disclosure, the product resulting from the reaction was subjected to extraction with DCM and H₂O. The organic layer thus formed was isolated, added with MgSO₄, and filtered.

In a particular embodiment of the present disclosure, the filtrate was recrystallized at a reduced pressure to afford cyclopentaphenanthrene-4-one. The recrystallization was achieved by adding dichloromethane (DCM) and hexane solution.

### Step (c): Synthesis of 2,2′-(((4H-Cyclopentaphenanthrene-4,4-Diyl)Bis (4,1-Phenylene)) Bis (Oxy)) Diethanol (Compound 1)

In a particular embodiment of the present disclosure, step (c) is set to react cyclopentaphenanthrene-4-one with phenoxyethanol to synthesize 2,2′-(((4H-cyclopentaphenanthrene-4,4-diyl) bis (4,1-phenylene)) bis (oxy)) diethanol, as illustrated in the following Reaction Scheme 1-3:

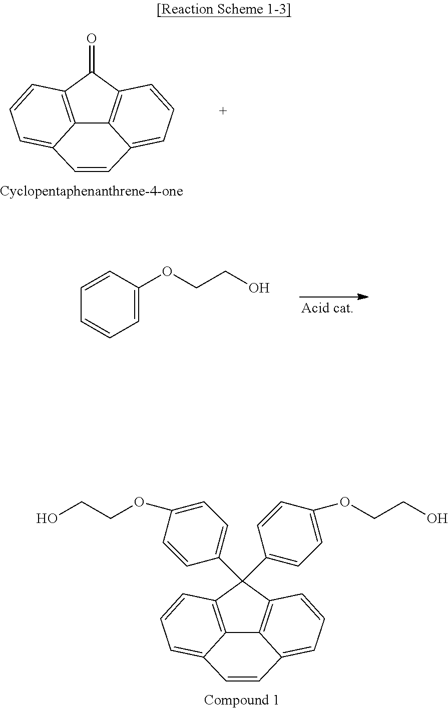 [SMILES: O=c1c2cccc3ccc4cccc1c4c32 ; OCCOc1ccccc1 ; OCCOc1ccc(C2(c3ccc(OCCO)cc3)c3cccc4ccc5cccc2c5c34)cc1]

In a particular embodiment of the present disclosure, the cyclopentaphenanthrene-4-one synthesized in step (b) was reacted with phenoxyethanol in the presence of an acid catalyst to synthesize 2,2′-(((4H-cyclopentaphenanthrene-4,4-diyl) bis (4,1-phenylene)) bis (oxy)) diethanol (Compound 1).

In a particular embodiment of the present disclosure, phenoxyethanol may be added in an amount of 20 to 80 mmol, 20 to 70 mmol, 20 to 60 mmol, 20 to 50 mmol, 20 to 40 mmol, 25 to 80 mmol, 25 to 70 mmol, 25 to 60 mmol, 25 to 50 mmol, 25 to 40 mmol, 30 to 80 mmol, 30 to 70 mmol, 30 to 60 mmol, 30 to 50 mmol, 30 to 40 mmol, 35 to 80 mmol, 35 to 70 mmol, 35 to 60 mmol, 35 to 50 mmol, 35 to 40 mmol, 20 mmol, 25 mmol, 30 mmol, 35 mmol, or 40 mmol, based on 10 mmol of the cyclopentaphenanthrene-4-one synthesized in step (b), but with no limitations thereto.

In a particular embodiment of the present disclosure, the acid catalyst may be an organic acid [sulfuric acid, hydrogen sulfate (alkali metal hydrogen sulfate such as sodium hydrogen sulfate, etc.), hydrogen chloride, hydrochloric acid (5-36 wt %, preferably 20-36 wt % hydrogen chloride solution, etc.), phosphoric acid, etc.] or an organic acid [sulfonic acid (alkane sulfonic acid such as methane sulfonic acid, etc.)]. The sulfuric acid used in the reaction may include diluted sulfuric acid (e.g., 30-90% sulfuric acid), concentrated sulfuric acid (e.g., 90% or higher sulfuric acid), and fuming sulfuric acid. In this regard, when the reaction system allows for conversion with sulfuric acid, sulfur trioxide may be used as a sulfuric acid precursor. Acid catalysts may be used alone or in combination. A preferable acid catalyst is hydrochloric acid or sulfuric acid.

The reaction may be performed using the acid catalyst in combination with thiols as a cocatalyst. When combined with thiols, the acid catalyst enables the condensation reaction to be carried out conveniently and effectively. So long as it functions as a cocatalyst, any thiol may be used. Examples of the thiol include mercaptocarboxylic acid (thioacetic acid, β-mercaptopropionic acid, α-mercaptopropionic acid, thioglycolic acid, thiooxalic acid, mercaptosuccinic acid, mercaptobenzoic acid, etc.), alkyl mercaptan (C1-16 alkyl mercaptan (particularly, C1-4 alkyl mercaptan) such as methyl mercaptan, ethylmercaptan, propyl mercaptan, isopropyl mercaptan, n-butyl mercaptan, dodecyl mercaptan, and so on), aralkyl mercaptan (benzyl mercaptan, etc.) or salts thereof. The salts may be exemplified by alkali metal salts (sodium salt, etc.). Preferred among the thiols is mercapto (especially, thioacetic acid and β-mercaptopropionic acid). Thiols may be used alone or in combination.

### Step (d): Synthesis of 4,4′-(4H-Cyclopentaphenanthrene-4,4-Diyl) Diphenol (Compound 2)

In step (d), 2,2′-(((4H-cyclopentaphenanthrene-4,4-diyl) bis (4,1-phenylene)) bis (oxy)) diethanol is reacted with potassium hydroxide to synthesize 4,4′-(4H-cyclopentaphenanthrene-4,4-diyl) diphenol as illustrated in the following Reaction Scheme 1-4:

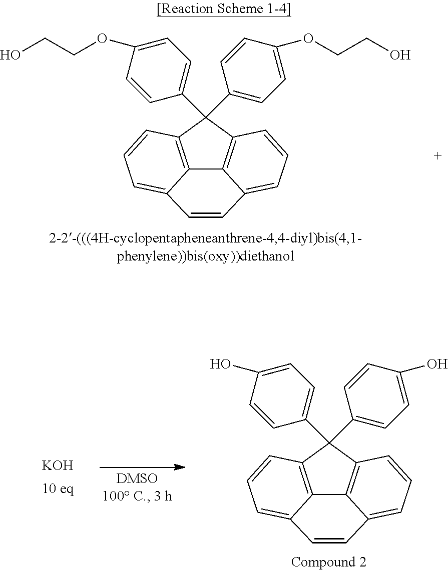 [SMILES: OCCOc1ccc(C2(c3ccc(OCCO)cc3)c3cccc4ccc5cccc2c5c34)cc1 ; "KOH" ; Oc1ccc(C2(c3ccc(O)cc3)c3cccc4ccc5cccc2c5c34)cc1]

In a particular embodiment of the present disclosure, 2,2′-(((4H-cyclopentaphenanthrene-4,4-diyl) bis (4,1-phenylene)) bis (oxy)) diethanol synthesized in step (c) is reacted with potassium hydroxide while stirring, to synthesize 4,4′-(4H-cyclopentaphenanthrene-4,4-diyl) diphenol (Compound 2).

In a particular embodiment of the present disclosure, potassium hydroxide (KOH) may be added in an amount of 15 to 60 mmol, 15 to 55 mmol, 15 to 50 mmol, 15 to 45 mmol, 15 to 40 mmol, 15 to 30 mmol, 15 to 25 mmol, 15 to 30 mmol, 20 to 60 mmol, 20 to 55 mmol, 20 to 50 mmol, 20 to 45 mmol, 20 to 40 mmol, 20 to 30 mmol, 20 to 25 mmol, 30 to 60 mmol, 30 to 55 mmol, 30 to 50 mmol, 30 to 45 mmol, 30 to 40 mmol, 20 mmol, 25 mmol, or 30 mmol, based on 3 mmol of 2,2′-(((4H-cyclopentaphenanthrene-4,4-diyl) bis (4,1-phenylene)) bis (oxy)) diethanol, but with no limitations thereto.

In a particular embodiment of the present disclosure, the reaction may be carried out in a nitrogen atmosphere.

In a particular embodiment of the present disclosure, the solvent for the reaction may be dimethylsulfoxide (DMSO), but is not limited thereto.

In a particular embodiment of the present disclosure, the reaction may be performed at a temperature of 80 to 150° C., 80 to 140° C., 80 to 130° C., 80 to 120° C., 80 to 110° C., 80 to 100° C., 90 to 150° C., 90 to 140° C., 90 to 130° C., 90 to 120° C., 90 to 110° C., 90 to 100° C., 100 to 150° C., 100 to 140° C., 100 to 130° C., 100 to 120° C., 100 to 110° C., 80° C., 90° C., 100° C., 110° C., or 120° C., but with no limitations thereto.

In a particular embodiment of the present disclosure, the reaction (stirring) may proceed for 1 to 6 hours, 1 to 5 hours, 1 to 4 hours, 1 to 3 hours, 2 to 6 hours, 2 to 5 hours, 2 to 4 hours, 2 to 3 hours, 3 to 6 hours, 3 to 5 hours, 3 to 4 hours, 1 hour, 2 hours, 3 hours, or 4 hours, but with no limitations thereto.

In a particular embodiment of the present disclosure, the reaction mixture after the reaction may be adjusted to have an acidic pH value. The pH may be 1 to 4, 1 to 3, or 1 to 2, but is not limited thereto.

In a particular embodiment of the present disclosure, after the reaction mixture was adjusted to have an acidic pH value, extraction with ethyl acetate, brine, and water was performed.

In a particular embodiment of the present disclosure, the organic layer this formed was isolated, added with MgSO₄, and filtered.

In a particular embodiment of the present disclosure, the filtrate was recrystallized at a reduced pressure to afford 4,4′-(4H-cyclopentaphenanthrene-4,4-diyl) diphenol (Compound 2). The recrystallization may be achieved using dichloromethane (DCM) and hexane solution.

According to another aspect of the present disclosure, the phenanthrene-based compound of the present disclosure possesses four phenyl groups, which can enhance or improve various properties, including optical characteristics. Therefore, these phenanthrene-based compounds can be usefully employed as components of resins, additives, etc. Moreover, because these phenanthrene-based compounds have multiple hydroxyl groups, they can efficiently improve the properties of resins when constituting resin components

In an embodiment of the present disclosure, the resin component may be (i) a resin that includes the phenanthrene-based compound represented by Chemical Formula 1 of the present disclosure as a monomer or (ii) a resin composed of the phenanthrene-based compound and another resin.

In an embodiment of the present disclosure, the resin as the resin component (either resin component (i) or (ii)) is not particularly limited and may include conventional thermoplastic resins and thermosetting resins (or photocurable resins). The resins as resin components may be used alone or in a combination.

Examples of the thermoplastic resin include olefin-resins (polyethylene, polypropylene, based polymethylpentene, amorphous polyolefin, etc.), halogen-containing vinyl-based resins (chlorine-containing resins such as polyvinyl chloride, fluorine-containing resins), acryl-based resins, styrene-based resins (polystyrene, acrylonitrile-styrene resin, etc.), polycarbonate-based resins (bisphenol A-type polycarbonate, etc.), polyester-based resins (polyalkylenearylate-based resins such as polyethylene terephthalate, polybutylene terephthalate, polycyclohexane dimethylene terephthalate, polyethylene naphthalate, etc., polyarylate-based resins, liquid crystal polyester, and the like), polyacetal-based resins, polyamide-based resins (polyamide 6, polyamide 66, polyamide 46, polyamide 6T, polyamide, MXD, etc.), polyphenylene ether-based resins (modified polyphenyleneether, etc.), polysulfone-based resins (polysulfone, polyethersulfone, etc.), polyphenylene sulfide-based resins (polyphenylene sulfide, etc.), polyimide-based resins (polyetherimide, polyamideimide, polyaminobismaleimide, bismaleimide triazine resin, etc.), polyether ketone-based resins (polyether ketone, polyetherether ketone, etc.), thermoplastic elastomers (polyamide-based elastomer, polyester-based elastomer, polyurethane-based elastomer, polystyrene-based elastomer, polyolefin-based elastomer, polydiene-based elastomer, polyvinyl chloride elastomer, and fluorine-based thermoplastic elastomer). The thermoplastic resins may be used alone or in combination.

Examples of the thermosetting resin include phenol resins, amino resins (urea resins, melamine resins, etc.), furan resins, unsaturated polyester-based resins, epoxy resins, thermosetting polyurethane-based resins, silicone resins, thermosetting polyimide-based resins, diallyl phthalate resins, vinyl ester resins (resins obtained by reacting epoxy resin with (meth) acrylic acid or a derivative thereof, and resins obtained by reacting polyhydric phenols with glycidyl (meth) acrylate, etc.). Within the scope of the thermosetting resin (or photocurable resin), polyfunctional (meth) acrylate, and vinyl ether resins (divinyl ether obtained by reacting a diol and acetylene) also fall. Thermosetting resins may be used alone or in combination.

Moreover, thermosetting resins may contain initiators, reactive diluents, curing agents, and accelerators according to type of thermosetting resins (or photocurable resins). For example, resin compositions including epoxy resins or urethane resins may contain amine-based curing agents while resin compositions including unsaturated polyester resins or vinyl ester resins may contain initiators (such as peroxides), polymerizable monomers (reactive diluents such as (meth) acrylate esters, styrene, etc.).

The resin components (i) or (ii) of the present disclosure may be used alone or in combination.

For the resin component (i) included as a constituent (monomeric component), the resin may preferably have the skeleton composed of the phenanthrene-based compound, and the resin component can be prepared using a phenanthrene-based compound corresponding to the polymer component (or monomeric component) as the polymer component or constituent (e.g., polyol such as diol, etc.) of the resin. For instance, resins employing a polyol component (especially diol component) as a polymer component or constituent (polyester-based resin, polyurethane-based resin, epoxy resin, vinyl ester resin, epoxy resin, multifunctional (meth) acrylate, (poly) urethane (meth) acrylate, (poly) ester (meth) acrylate, vinyl ether, etc.) may preferably contain the phenanthrene-based compounds for entirety or part of the polyol component.

In the resin component (i), the phenanthrene-based compound may be used alone or in combination with two or more types thereof as a polymer component (or constituent).

Preferred resins constituting the resin component (or resin components) include polyester resins, polyurethane-based resins (thermoplastic or thermosetting polyurethane resins), polycarbonate-based resins, acrylic resins [including thermosetting or photocurable resins with multifunctional (meth) acrylates], epoxy resins, vinyl ethers, etc. Additionally, aromatic ring (benzene ring)-containing resins (thermoplastic resins), such as aromatic polycarbonate resins (e.g., bisphenol A-type polycarbonate), polyester resins [polyalkylene arylate resins; and polyarylate resins using aromatic dicarboxylic acids (terephthalic acid, etc.) and aromatic diols (biphenol, bisphenol A, xylylene glycol, and alkylene oxide adducts thereof) as polymer components, etc.], polysulfone resins (polysulfone, polyethersulfone, etc.), polyphenylene sulfide resins (polyphenylene sulfide, etc.) are also desirable.

Below, resins (or resin component (i)) containing phenanthrene-based compound represented by Chemical Formula 1 as a monomeric component (polymer component, constituent, copolymer component) will be explained for representative resins (or resin components).

### (1) Polyester-Base Resin

Polyester-based resins containing the phenanthrene-based compound as a polymer component (particularly, polyester-based resin polymerized from the phenanthrene-based compound (generally, compound with p1=p2=1) as a monomeric component) can be obtained by reacting the phenanthrene-based compound and a dicarboxylic acid component. Within the scope of the polyester-based resin, polyarylate-based resins using aromatic dicarboxylic acid as a polymer component as well as saturated or unsaturated polyester-based resins also fall.

The polyol component (particularly diol component) in polyester-based resins may be constituted of different diol components different the phenanthrene-based compound. Examples of such diol components (or diols) include alkylene glycol (e.g., linear or branched C2-12 alkylene glycols, such as ethyleneglycol, propylene glycol, trimethylene glycol, 1,3-butanediol, tetramethyleneglycol, hexanediol, neopentylglycol, octanediol, decanediol, etc.), (poly) oxyalkylene glycol (e.g., diethyleneglycol, triethylene glycol, dipropyleneglycol, C2-4 alkylene glycol, etc.), aliphatic diol (e.g., 1,4-cyclohexanediol, 1,4-cyclohexanedimethanol, 2,2-bis (4-hydroxycyclohexyl), propane or alkylene oxide adducts thereof (2,2-bis (4-(2-hydroxyethoxy) cyclohexyl) propane, etc.)), aromatic diols (e.g., biphenol, 2,2-bis (4-hydroxyphenyl) propane (bisphenol A), bisphenol AD, bisphenol F, or alkylene oxides (C2-3 alkylene oxide) adducts thereof (2,2-bis (4-(2-hydroxyethoxy) phenyl) propane, etc.), xylene glycol, etc.). Their diols may be used alone or in combination.

Preferable diols include linear or branched C2-10 alkylene glycol, especially C2-6 alkylene glycols (e.g., linear or branched C2-4 alkylene glycols such as ethylene glycol, propylene glycol, 1,4-butanediol, etc.). For the most part, at least ethylene glycol is used as the diol. Such diols (e.g., ethylene glycol) can be used to enhance polymerization reactivity while imparting flexibility to the resin.

The phenanthrene-based compounds and the diols may be used at a rate (molar ratio) ranging from 100/0 to 50/50, preferably from 100/0 to 75/25 (e.g., 100/0 to 70/30), and more preferably from 100/0 to 90/10 (e.g., 100/0 to 80/20).

The diol component may be used in combination with a polyol such as glycerin, trimethylol propane, trimethylol ethane, pentaerythritol, etc., as necessary.

The dicarboxylic acid component as a constituent of the polyester-based resins is exemplified by aliphatic dicarboxylic acid, cyclic dicarboxylic acid, aromatic dicarboxylic acid, and their derivatives capable of forming ester [e.g., acid anhydride; acid halide (acid chloride); and lower alkyl ester (C1-2 alkyl ester, etc.)]. The dicarboxylic acids thereof may be used alone or in combination.

Available as the aliphatic dicarboxylic acid may be saturated C3-20 aliphatic dicarboxylic acid (preferably, saturated C3-14 aliphatic dicarboxylic acid, etc.), succinic acid, glutaric acid, adipic acid, pimelic acid, suberic acid, azelaic acid, sebasic acid, dodecane dicarboxylic acid, hexadecane dicarboxylic acid, etc.; unsaturated C4-20 aliphatic dicarboxylic acid (preferably, unsaturated C4-14 aliphatic dicarboxylic acid) such as maleic acid, fumaric acid, citraconic acid, mesaconic acid; and their derivatives capable of forming ester. The ratio of the aliphatic unsaturated dicarbonic acid (maleic acid or an anhydride thereof) in the unsaturated polyester-based resin, may range from about 10 to 100 mol %, preferably from about 30 to 100 mol %, and more preferably from 50 to 100 mol % (e.g., 75 to 100 mol %), based on the entire dicarboxylic acid component.

The cyclic dicarboxylic acid can be exemplified by saturated cyclic dicarboxylic acid (e.g., C3-10 cycloalkane-dicarboxylic acid such as cyclopentane dicarbonic acid, 1,4-cyclohexane dicarboxylic acid, 1,3-cyclohexane dicarboxylic acid, 1,2-cyclohexane dicarboxylic acid, cycloheptane dicarboxylic acid, etc.), unsaturated cyclic dicarboxylic acid (e.g., C3-10 cycloalkene dicarboxylic acid such as 1,2-cyclohexene dicarboxylic acid, 1,3-cyclohexene dicarboxylic acid, etc.); polycyclic alkane dicarboxylic acids (di- or tricyclo C7-10 alkane-dicarboxylic acid such as borane dicarbonic acid, norbornane dicarboxylic acid, adamantane dicarboxylic acid, etc.), polycyclic alkene dicarboxylic acids (di- or tricyclo C7-10 alkene dicarboxylic acid such as bornene dicarboninic acid, norbornene dicarboxylic acid, etc.), and their derivatives capable of forming ester.

Examples of the aromatic dicarboxylic acid include aromatic C8-16 dicarboxylic acids such as phthalic acid, isophthalic acid, terephthalic acid, naphthalene dicarboxylic acid (2,6-naphthalene dicarboxylic acid, etc.), 4,4′-diphenyldicarboxylic acid, diphenyl ether-4,4′-dicarboxylic acid, 4,4′-diphenylmethane dicarboxylic acid, 4,4′-diphenylketone dicarboxylic acid, etc.; and their derivatives capable of forming ester.

The dicarboxylic acids may be used in combination with polyhydric carboxylic acid such as trimellitic acid, pyromellitic acid, etc., as necessary.

As the dicarboxylic acid component, at least one selected from aliphatic dicarboxylic acid and cyclic dicarboxylic acid may be generally used. Particularly preferable is aliphatic dicarboxylic acid (e.g., saturated aliphatic dicarboxylic acid or its derivatives capable of forming ester, especially saturated C3-14 aliphatic dicarboxylic acid such as adipic acid, suberic acid, sebasic acid, etc.) or cyclic dicarboxylic acid (C5-10 cycloalkane dicarboxylic acid such as cyclohexane dicarboxylic acid, etc.).

For the polyarylate-based resin, a dicarboxylic acid component including at least one aromatic dicarboxylic acid is used. The aromatic dicarboxylic acid may be used in combination with a different dicarboxylic acid (aliphatic dicarboxylic acid and/or cyclic dicarboxylic acid). The aromatic dicarboxylic acid and a different dicarboxylic acid may be used at a molar ratio from 100/0 to 10/90, preferably from 100/0 to 30/70, and more preferably from 100/0 to 50/50.

In the polyester-based resin, the dicarboxylic acid component and the polyol component (diol component, the phenanthrene-based compound, etc.) may be present at a rate (molar ratio) from 1.5/1 to 0.7/1 and preferably from 1.2/1 to 0.8/1 (particularly 1.1/1 to 0.9/1).

The polyester-based resin may have a weight average molecular weight (Mw, polystyrene conversion) of, for example, 100 to 50×10⁴, preferably 500 to 30×10⁴ (e.g., 1000 to 20×10⁴), and more preferably 3000 to 30×10⁴, but with no limitations thereto. In addition, the unsaturated polyester-based resin may have a molecular weight of about 300 to 1,000, preferably about 350 to 800, and more preferably about 400 to 700, per double bond. The terminal group in the polyester-based rein may be a hydroxyl group or carboxyl group and may be protected with a protecting group if necessary.

The polyester-based resin can be prepared using a typical method, for example, direct polymerization method (direct esterification) or ester exchange method, for condensing the polyol component composed of the phenanthrene-based compound (especially diol component) and the dicarboxylic acid component.

### (2) Polyurethane Resin

The polyol component (diol component) as a constituent of a polyurethane-based resin containing the phenanthrene-based compound as a polymer component (monomeric component) may be composed of the phenanthrene-based compound alone or in combination with the diols exemplified for the polyester-based resin. In addition, a polyester diol produced by reaction between diol a component containing the phenanthrene-based compound as a constituting unit, for example, a diol component composed of the phenanthrene-based compound of Chemical Formula 1 with p1=p2=1, and a dicarboxylic acid component, and a polyether diol produced by reaction between the diol component and alkylene oxide are also used as diol components for polyurethane-based resin. The diol components may be used alone or in combination. If necessary, the diol component may be used in combination with a polyol component such as triol, etc.

The content of the phenanthrene-based compound in the polyol component (diol component) may be 10-100 mol %, preferably 20-80 mol %, and more preferably 30-70 mol %, based on the total mole of the polyol component (diol component). Examples of the diisocynate compound as a component of polyurethane-based resins include aromatic diisocyanates [paraphenylenediisocyanate, tolylene diisocyanate (TDI), xylene diisocyanate (XDI), tetramethyl xylene diisocyanate (TMXDI), naphthalene diisocyanate (NDI), bis (isocyanatophenyl) methane (MDI), toluidine diisocyanate (TODI), 1,2-bis (isocyanatophenyl) ethane, 1,3-bis (isocyanatophenyl) propane, 1,4-bis (isocyanatophenyl) butane, polymeric MDI, etc.], cyclic diisocyanates [cyclohexane 1,4-diisocyanate, isophorone diisocyanate (IPDI), hydrogenated XDI, hydrogenated MDI, etc.], aliphatic diisocyanates [hexamethylenediisocyanate (HDI), trimethyl hexamethylenediisocyanate (TMDI), lysine diisocyanate (LDI), etc.]. These diisocyanate compounds may be used alone or in combination. If necessary, the diisocyanate compounds may be used in combination with polyisocyanate compounds (e.g., aliphatic triisocyanates such as 1,6,11-undecanetriisocyanate methyloctane, 1,3,6-hexamethylene triisocyanate, etc.; triisocyanates including cyclic triisocyanate such as bycycloheptane triisocyanate, etc.), monoisocyanate compounds (C1-6 alkyl isocyanate such as methyl isocyanate, etc.; C5-6 cycloalkyl isocyanate such as cycloalkyl isocyanate, etc.; and C6-10 aryl isocyanate, such as phenyl isocyanate, etc.). The isocyanate compounds encompass derivatives thereof, such as multimers or modified compounds thereof.

The polyurethane-based resins may be prepared using a typical urethane method in which, for example, a diisocyanate component is used in an amount of 0.7-2.5 moles, preferably 0.8-2.2 moles, and more preferably 0.9-2 moles per mole of a polyol component (diol component). In addition, based on one mole of a diol component, the diisocyanate component can be used in an amount of 0.7-1.1 moles to afford a theramoplastic resin and in an excess amount (e.g., about 1.5-2.2 moles) to afford a thermosetting resin with a free terminal isocyanate group.

### (3) Polycarbonate-Based Resin

Polycarbonate-based resins containing the phenanthrene-based compound as a polymer component may be obtained using a typical method in which, for example, a polyol component (particularly diol component) composed of the phenanthrene-based compound (generally, the compound of Chemical Formula 1 with p1=p2=1) is reacted with phosgene (phosgene method) or with ester carbonate (transesterification method).

The polyol component (diol component) may be composed of the phenanthrene-based compound alone or in combination with the diols (diols exemplified for the polyester-based resin, especially aromatic diols or cyclic diols). Other diols may be used alone or in combination. Preferable among the other diols are aromatic diols including bisphenols such as bisphenol A, AD, F, etc. The ratio between the hydroxyl-containing phenanthrene-based compound and the diols may be selected from the same range as for the polyester-based resins.

The polycarbonate-based resin may have a molecular weight, for example, weight average molecular weight of about 1×10³ to 100×10⁴ (e.g., 1×10⁴ to 100×10⁴), preferably 5×10³ to 50×10⁴ (e.g., 1×10⁴ to 50×10⁴), and more preferably 1×10⁴ to 25×10⁴ (e.g., 1×10⁴-10×10⁴), but with no limitations thereto.

### (4) Epoxy-Based Resin

The diol or polyol component as a constituent of epoxy-based resin may be composed of the phenanthrene-based compound (generally, the compound of Chemical Formula 1 with p1=p2=1) alone or in combination with diols (particularly aromatic diols or aliphatic diols) different from those for the polyester-based resin. The different diols may be used alone or in combination. Preferable among the diols are aromatic diols including bisphenols such as bisphenol A, AD, F, and so on. The ratio between the hydroxyl-containing phenanthrene-based compound and the diols may be selected from the same range as for the polyester-based resins. Furthermore, the diols different from the bisphenolphenanthrene-based compounds may be used in combination with polyols (e.g., phenol novolac), as needed.

The epoxy resin may be obtained by reacting the phenanthrene-based compound with epichlorohydrin. The epoxy-based resins may have a weight average molecular weight (Mw) of, for example, 300 to 30,000, preferably 400 to 10,000, and more preferably 500 to 5,000.

### (5) Vinyl Ester-Based Resin

Vinyl ester-based resins can be obtained using a typical method in which, for example, the epoxy resin (having the phenanthrene-based compound as a constituent) is reacted with a polymerizable monomer possessing a carboxyl group (unsaturated monocarboxylic acid). The polymerizable monomer with a carboxylic acid group may be used in combination with dicarboxylic acid (aliphatic dicarboxylic acid, cyclic dicarboxylic acid, or aromatic dicarboxylic acid (isophthalic acid, terephthalic acid, etc.)) of the polyester-based resin as necessary.

As the polymerizable monomer with a carboxylic acid group, an unsaturated monocarboxylic acid may be used. The unsaturated acid may generally include Available are also cinnamic acid, (meth) acrylic acid, crotonic acid, sorbic acid, maleic acid monoalkyl ester (monomethyl maleate, etc.). These monomers may be used alone or in combination.

Unsaturated monocarboxylic acid may be used in an amount of 0.5-1.2 moles, preferably 0.7-1.1 moles, and more preferably 0.8-1 mole per mole of the epoxy group in the epoxy resin.

The vinyl ester-based resin can be obtained by reacting the phenanthrene-based compound with glycidyl (meth) acrylate. Glycidyl (meth) acrylate may be used in an amount of 1-3 moles and preferably 1-2 moles per mole of the phenanthrene-based compound.

### (6) Acrylic Resin

The monomer of acrylic resin may be obtained by reacting the phenanthrene-based compound with a carboxylic acid group-bearing polymerizable monomer. As the polymerizable monomer with a carboxylic acid group, unsaturated monocarboxylic acid, particularly (meth) acrylic acid may be used. Available are cinnamic acid, crotonic acid, sorbic acid, maleic acid, monoalkylester (monomethyl maleate, etc.). In addition, reactive derivatives, such as acid chloride, C1-2 alkyl ester, etc., may be used instead of the unsaturated carboxylic acid. These monomers may be used alone or in combination.

Acrylic resin may be a homo- or copolymer of (meth) acrylic monomers having the phenanthrene-based skeleton, or a copolymer of an acrylic monomer having the phenanthrene-based skeleton and a different polymeric monomer. Examples of the copolymerizable monomer include: carboxylic acid group-bearing monomers such as (meth) acrylic acid, maleic acid, maleic anhydride, etc.; (meth) acrylic acid ester [(meth) acrylic acid C1-6 alkyl ester such as (meth) acrylic acid methyl, etc.]; vinyl cyanides such as (meth) acrylonitrile, etc.; aromatic vinyl monomers such as styrene, etc.; vinylester carboxylates, such as vinyl acetate, etc.; α-olefins such as ethylene, propylene, etc.

These copolymerizable monomers may be used alone or in combination.

In addition, a monomer possessing a plurality of (meth) acryloyl groups obtained by a reaction between the phenanthrene-based compound and a carboxylic acid-bearing polymerizable monomer may be used as an acrylic resin (that is, thermosetting acrylic resin, and oligomer (resin precursor)).

The resin component (ii) can be prepared or obtained by mixing the phenanthrene-based compound and a resin (optionally additive).

The mixing method is not particularly limited and can include melt compounding methods using such as mixers like ribbon blenders, tumble mixers, Henschel mixers, or compounding equipment like open rollers, kneaders, Banbury mixers, extruders, etc. These mixing methods can be used alone or in combination.

Additionally, the proportion of the phenanthrene-based compound in the resin component (ii) may be, for example, 1 to 80 parts by weight, preferably 5 to 60 parts by weight, and more preferably 20 to 60 parts by weight, per 100 parts by weight of the resin.

The resin component may include additives. Due to the phenanthrene-based skeleton derived from the phenanthrene-based compound, the dispersion of additives can be improved.

Additives may be liquid at room temperature (for example, around 15-25° C.) or in solid form (for example, granular solids). Additives s may include fillers or reinforcing agents, colorants (dyes and pigments), conductive agents, flame retardants, plasticizers, lubricants, stabilizers (antioxidants, UV absorbers, heat stabilizers, etc.), mold release agents (natural waxes, synthetic waxes, straight-chain fatty acids or their metal salts, acid amides, esters, paraffins, etc.), antistatic agents, dispersants, flow modifiers, leveling agents, blowing agents, surface modifiers (such as silane coupling agents or titanium-based coupling agents), stress relievers (silicone oils, silicone rubbers, various plastic powders, various high-performance plastic powders, etc.), heat resistance improvers (sulfur compounds or polysilanes), carbon materials, etc. These additives can be used alone or in combination.

Among these additives, fillers, colorants (such as black pigments, red pigments, green pigments, blue pigments, etc.), flame retardants, and carbon materials are preferred. Carbon materials functioning as fillers or reinforcing agents, colorants, conductive agents, etc., are also desirable.

The resin component can be formed into molded articles by known molding methods depending on the form (resin pellets, coating compositions, etc.), such as injection molding, injection compression molding, extrusion molding, transfer molding, blow molding, compression molding, and coating methods (spin coating, roll coating, curtain coating, dip coating, casting molding, etc.). The shape of the molded articles can include two-dimensional structures (films, sheets, coatings (or thin films), plates, etc.) and three-dimensional structures (for example, tubes, rods, tubes, leather, hollow products, etc.).

Provided in the present disclosure are a high-refractive-index monomer and a preparation method therefor, wherein the monomer has a phenanthrene-based complex cardo structure and can be used in optical resins requiring a refractive index of 1.7 or higher. Due to the phenanthrene-based complex cardo structure of the compound, the fluidity of the molecular chains can be maximally suppressed, allowing for the production of materials such as resins with excellent thermal stability, characterized by a high glass transition temperature.

A better understanding of the present disclosure may be obtained through the following examples which are set forth to illustrate, but are not to be construed to limit, the present disclosure.

### EXAMPLES

Throughout the description, the term “%” used to express the concentration of a specific material, unless otherwise particularly stated, refers to (wt/wt) % for solid/solid, (wt/vol) % for solid/liquid, and (vol/vol)% for liquid/liquid.

### EXAMPLE 1: Synthesis of Pyrene-4,5-Dione

In a two-neck flask equipped with a thermometer and a stirrer, flame drying was conducted with a magnetic bar. Pyrene (4.09 g, 20 mmol) and RuCl (0.414 g, 2 mmol) were introduced and maintained under a nitrogen atmosphere. THE (100 mL) was added and stirred. In an Erlenmeyer flask, NaIO₄ (18.822 g, 88 mmol) was added to and mixed with H₂O (150 mL). The aqueous NaIO₄ solution was slowly added to the two-neck flask while stirring. Then, dichloromethane (DCM) (100 mL) was added and stirred at room temperature for 5 hours. The solids thus formed were filtered out. The filtrate was subjected to extraction with dichloromethane and H₂O. The organic layer was isolated, added with MgSO₄, and filtered. Then, this filtrate was recrystallized in dichloromethane and hexane at a reduced pressure to afford pyrene-4,5-dione (3.03 g, yield 65.0%).

### EXAMPLE 2: Synthesis of Cyclopentaphenanthrene-4-One

After a reflux tube was installed in a two-neck flask equipped with a thermometer and a stirrer, the pyrene-4,5-dione (2.34 g, 10 mmol) prepared in Example 1 was introduced into the flask and maintained under an oxygen atmosphere. In a flask, H₂O (50 mL) and NaOH (8.0 g, 20 mmol) were mixed to prepare an aqueous NaOH solution. This NaOH solution was slowly added to the two-neck flask, followed by stirring at 90° C. for 2 days. Extraction with DCM and H₂O was conducted. The organic layer thus formed was isolated, added with MgSO₄, and filtered. The filtrate was recrystallized in dichloromethane and hexane at a reduced pressure to afford cyclopentaphenanthrene-4-one) (1.12 g, yield 55.0%).

### EXAMPLE 3: Synthesis of 2,2′-(((4H-Cyclopentaphenanthrene-4,4-diyl) bis (4,1-phenylene)) bis (oxy)) diethanol (Compound 1)

In a two-neck flask equipped with a thermometer and a stirrer, flame drying was conducted with a magnetic bar. Cyclopentaphenanthrene-4-one (2.04 g, 10 mmol) prepared in Example 2 was introduced and maintained under a nitrogen atmosphere. Phenoxyethanol (5.02 mL, 40 mmol) and mercaptopropionic acid (100 uL, 1.0 mmol) were added and stirred at 50° C. for 10 minutes. H₂SO₄ (320 uL, 30 mmol) was dropwise added. At the same temperature, stirring was conducted for 5 hours. Extraction was made with DCM and H₂O. The organic layer thus formed was isolated, added with MgSO₄, and filtered. The filtrate was recrystallized in ethyl acetate and hexane at a reduced pressure to afford Compound 1 as a white solid (3.25 g, yield 70.0%, purity 100.0%). ¹H NMR (400 MHZ, CDCl₃) δ 7.87 (s, 2H), 7.83 (d, J=0 Hz, 2H), 7.81-7.59 (m, 2H), 7.57 (t, J=4 Hz, 2H), 7.19 (d, J=8 Hz, 4H), 6.77 (d, J=8 Hz, 4H), 4.01 (dd, J=4, 4 Hz, 4H), 3.9 (d, J=4 Hz, 4H)

According to Example 3, 2,2′-(((4H-cyclopentaphenanthrene-4,4-diyl) bis (4,1-phenylene)) bis (oxy)) diethanol was found to be synthesized with high purity.

### EXAMPLE 4: Synthesis of 4,4′-(4H-Cyclopentaphenanthrene-4,4-diyl) diphenol (Compound 2)

In a two-neck flask equipped with a thermometer and a stirrer, flame drying was conducted with a magnetic bar. 2,2′-(((4H-Cyclopentaphenanthrene-4,4-diyl) bis (4,1-phenylene)) bis (oxy)) diethanol (1.4 g, 3 mmol) and KOH (1.7 g, 30 mmol) were introduced and maintained under a nitrogen atmosphere. DMSO (20 mL) was added and stirred at 100° C. for 3 hours. After stirring, 1 M HCl was added until the pH was adjusted to 2. Extraction was made with ethyl acetate, brine, and water. The organic layer thus formed was isolated, added with MgSO₄, and filtered. The filtrate was recrystallized in ethyl acetate and hexane at a reduced pressure to afford Compound 2 as a white solid (0.99 g, yield 88.0%, purity 99.4%). ¹H NMR (400 MHZ, CDCl₃) δ 7.85 (s, 2H), 7.80 (d, J=8 Hz, 2H), 7.62 (t, J=8 Hz, 2H), 7.56 (d, J=4 Hz, 2H), 7.16 (t, J=14 Hz, 4H), 6.70 (dd, J=8, 36 Hz, 4H)

According to Example 4, 4,4′-(4H-cyclopentaphenanthrene-4,4-diyl) diphenol was found to be synthesized with high purity.

What is claimed is:

1. A phenanthrene-based compound having the chemical structure represented by the following Chemical Formula 1: 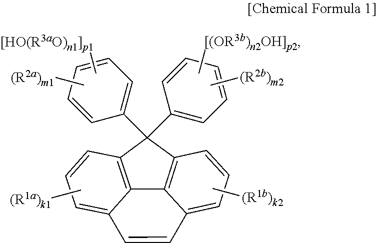
wherein R^{1a}, R^{1b}, R^{2a}, and R^{2b} are each a substituent bonded to the phenanthrene or phenyl moieties,
R^{3a} and Rb are each an alkylene;
k1 and k2 are each independently an integer of 0 to 4;
m1 and m2 are each independently an integer of 0 to 3;
n1 and n2 are each independently an integer of 0 or 1 or higher;
p1 and p2 are each independently an integer of 1 or higher,
with a proviso that m1+p1 and m2+p2 are each independently an integer of 1 to 4.

2. The phenanthrene-based compound of claim 1, wherein R^{3a} and Rob are each a C2 to C4 alkylene; and n1 and n2 are each 0 or 1.

3. The phenanthrene-based compound of claim 1, wherein p1 and p2 are each 1.

4. The phenanthrene-based compound of claim 1, wherein R^{1a} and R^{1b} are each independently a C1 to C4 alkyl; k1 and k2 are each independently 0 or 1; R^{2a} and R^{2b} are each independently a C1 to C4 alkyl, a C1 to C4 alkoxy, or a C6 to C8 aryl; and m1 and m2 are each independently an integer of 0 to 2.

5. The phenanthrene-based compound of claim 1, having the chemical structure of the following Chemical Formula 2-1 or 2-2:

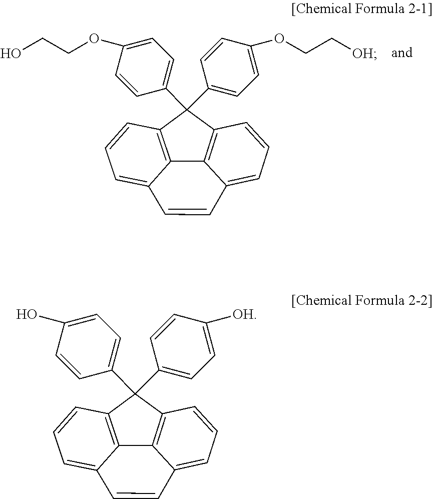 [SMILES: OCCOc1ccc(C2(c3ccc(OCCO)cc3)c3cccc4ccc5cccc2c5c34)cc1 ; Oc1ccc(C2(c3ccc(O)cc3)c3cccc4ccc5cccc2c5c34)cc1]

6. A method for preparing a phenanthrene-based compound, the method comprising the steps of:
(a) oxidizing pyrene to synthesize pyrene-4,5-dione;
(b) synthesizing cyclopentaphenanthrene-4-one from pyrene-4,5-dione; and
(c) synthesizing 2,2′-(((4H-cyclopentaphenanthrene-4,4-diyl) bis (4,1-phenylene)) bis (oxy)) diethanol from cyclopentaphenanthrene-4-one.

7. The method of claim 6, further comprising a step of (d) synthesizing 4,4′-(4H-cyclopentaphenanthrene-4,4-diyl) diphenol from 2,2′-(((4H-cyclopentaphenanthrene-4,4-diyl) bis (4,1-phenylene)) bis (oxy)) diethanol.

8. The method of claim 6, wherein the step (a) is set to oxidize pyrene with sodium periodate to synthesize pyrene-4,5-dione as illustrated by the following Reaction Scheme 1-1:

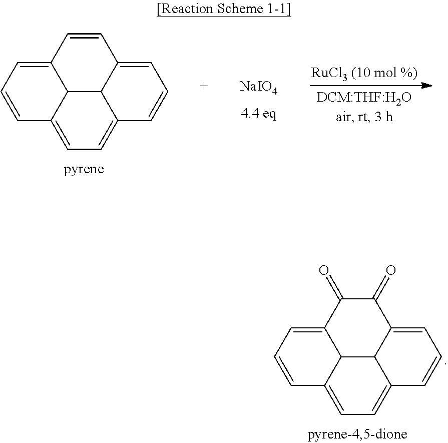

9. The method of claim 6, wherein the step (b) is set to react pyrene-4,5-dione with sodium hydroxide to synthesize cyclopentaphenanthrene-4-one as illustrated by the following Reaction Scheme 1-2:

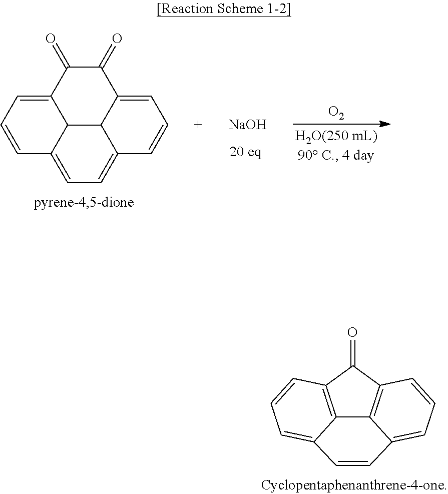 [SMILES: O=C1C(=O)C2=CC=CC3=CC=C4C=CC=C1C4C32 ; "NaOH" ; "O2" ; O=c1c2cccc3ccc4cccc1c4c32]

10. The method of claim 6, wherein the step (c) is set to react cyclopentaphenanthrene-4-one with phenoxyethanol to synthesize 2,2′-(((4H-cyclopentaphenanthrene-4,4-diyl) bis (4,1-phenylene)) bis (oxy)) diethanol as illustrated by the following Reaction Scheme 1-3:

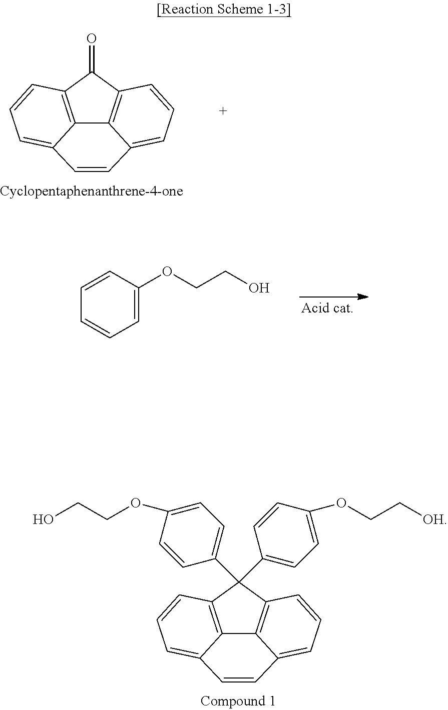 [SMILES: O=c1c2cccc3ccc4cccc1c4c32 ; OCCOc1ccccc1 ; OCCOc1ccc(C2(c3ccc(OCCO)cc3)c3cccc4ccc5cccc2c5c34)cc1]

11. The method of claim 6, wherein the step (d) is set to react 2,2′-(((4H-cyclopentaphenanthrene-4,4-diyl) bis (4,1-phenylene)) bis (oxy)) diethanol with potassium hydroxide to synthesize 4,4′-(4H-cyclopentaphenanthrene-4,4-diyl) diphenol as illustrated by the following Reaction Scheme 1-4: 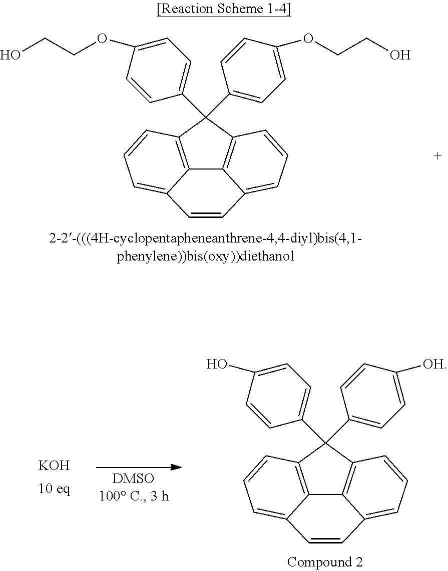 [SMILES: OCCOc1ccc(C2(c3ccc(OCCO)cc3)c3cccc4ccc5cccc2c5c34)cc1 ; "KOH" ; Oc1ccc(C2(c3ccc(O)cc3)c3cccc4ccc5cccc2c5c34)cc1]
